(19) United States
(12) Patent Application Publication
(10) Pub. No.: US 2024/0241114 A1
(43) Pub. Date: Jul. 18, 2024

(54) ANALYSIS KIT AND ANALYSIS METHOD

(71) Applicant: TDK CORPORATION, Tokyo (JP)

(72) Inventors: Takeshi SAKAMOTO, Tokyo (JP); Kyung-Ku CHOI, Tokyo (JP)

(73) Assignee: TDK CORPORATION, Tokyo (JP)

(21) Appl. No.: 18/433,865

(22) Filed: Feb. 6, 2024

Related U.S. Application Data
(62) Division of application No. 16/626,778, filed on Dec. 26, 2019, now abandoned.

(30) Foreign Application Priority Data
Jun. 30, 2017 (JP) 2017-129440
Mar. 30, 2018 (JP) 2018-069837

(51) Int. Cl. G01N 33/543; B01L 3/00; G01N 27/327; G01N 27/42; G01N 33/553

(52) U.S. Cl. CPC G01N 33/5438; B01L 3/50; G01N 27/3276; G01N 27/42; G01N 33/54326; G01N 33/553; B01L 2300/0645

(57) ABSTRACT

This analysis kit includes a sensor having a working electrode, a reference electrode and a counter electrode, primary antibodies being fixed to a surface of the working electrode of the sensor, and a dispersion liquid of magnetic metal nanoparticles including solvent and magnetic metal nanoparticles dispersed in the solvent, secondary antibodies being fixed to surfaces of the magnetic metal nanoparticles.

This application is a Divisional of application Ser. No. 16/626,778, filed Dec. 26, 2019, which is a National Stage of PCT Application No. PCT/JP2018/024860, filed Jun. 29, 2018, which claims priority to JP 2017-129440, filed Jun. 30, 2017, and JP 2018-069837, filed Mar. 30, 2018. The entire contents of the prior applications are hereby incorporated by reference herein in their entirety.

### TECHNICAL FIELD

The present invention relates to an analysis kit and an analysis method for analyzing for a test substance using an antigen-antibody reaction.

Priority is claimed on Japanese Patent Application No. 2017-129440, filed Jun. 30, 2017 and Japanese Patent Application No. 2018-069837, filed Mar. 30, 2018, the content of which is incorporated herein by reference.

### BACKGROUND ART

Detection of biological materials is performed in fields such as medical care, health care, and the environment. Additionally, it is desired to develop analysis methods capable of selectively quantifying a biological material to be measured in a plurality of biological materials with high sensitivity and simple operability.

As one of methods capable of selectively measuring a minute amount of biological material in a liquid with high sensitivity, an immunoassay method is known. The immunoassay method is a method for quantifying an antigen using a reaction (an antigen-antibody reaction) between a biological material (an antigen, a hapten, or the like) to be measured and a material (antibodies) which is bound to the antigen.

As a method for quantifying an antigen, a sandwich method is known. The sandwich method is a method of putting (sandwiching) an antigen between a solid to which primary antibodies are fixed and a label to which secondary antibodies are fixed. That is, the sandwich method is a method in which an antigen is captured with the primary antibodies, the captured antigen is bound to the secondary antibodies, and a label fixed to the secondary antibodies bound to the antigen is quantified. As a method for quantifying the label, a method in which metal particles are used as the label and an amount of the metal particles is quantified using an electrochemical method is known. In addition, since the antigen and the antibodies do not have electrical conductivity, it is difficult to quantify the label (the metal particles) bound to the antigen and the antibodies directly using an electrochemical method.

Patent Literature 1 discloses a diagnostic kit including at least one reagent labeled with colloidal metal particles, at least one electrode, and also a reagent for chemically dissolving the colloidal metal particles. In the diagnostic kit disclosed in Patent Literature 1, the colloidal metal particles as a label are chemically dissolved, the metal solution is then transferred to the electrode and reduced, the reduced metal is deposited on the electrode, the metal deposited on a surface of the electrode is electrically re-dissolved, and an amount of the metal is measured by analyzing a voltammetric peak which appears after the re-dissolution.

### CITATION LIST

### Patent Literature

[Patent Literature 1]
   Published Japanese Translation No. 2004-512496 of the PCT International Publication for patent application

### SUMMARY OF INVENTION

### Technical Problem

A method in which metal particles are used as a label and an amount of the metal particles is quantified using an electrochemical technique is a useful method from the viewpoint of sensitivity and accuracy. However, in the method for quantifying colloidal metal particles described in Patent Literature 1, since a process of chemically dissolving the colloidal metal particles, or the like is required, an operation is complicated, it takes time to obtain analysis results, and thus it is difficult to introduce the method in clinical examinations at medical sites.

The present invention has been made in view of the above problems, and an object thereof is to provide an analysis kit and an analysis method which are easy to operate and allow analysis for a test substance with high selectivity and high sensitivity.

### Solution to Problem

The inventors have found that it is possible to quantify magnetic metal nanoparticles without chemically dissolving them by binding an antigen (a test substance) to primary antibodies using a sensor having a specific working electrode to which primary antibodies are fixed and magnetic metal nanoparticles to which secondary antibodies are fixed, fixing the antigen to a working electrode, then binding the antigen with a secondary antigen, causing the magnetic metal nanoparticles to which the secondary antibodies bound to the antigen are fixed to come into close contact with the working electrode using a magnetic field, and ionizing (oxidizing) the magnetic metal nanoparticles in close contact with the working electrode using an electrochemical method. Additionally, the inventors have confirmed that an amount of current generated when the magnetic metal nanoparticles are ionized by the electrochemical method and an amount of antigen have a high correlation and that it is possible to determine the amount of antigen from the amount of current, and have completed the present invention.

That is, the present invention provides the following means to solve the problems.

(1) An analysis kit according to a first aspect includes a sensor having a working electrode, a reference electrode and a counter electrode, primary antibodies being fixed to a surface of the working electrode of the sensor; and a dispersion liquid of magnetic metal nanoparticles including solvent and magnetic metal nanoparticles dispersed in the solvent, secondary antibodies being fixed to surfaces of the magnetic metal nanoparticles.

(2) In the analysis kit according to the above-described aspect, the working electrode may be a carbon electrode, a metal electrode, a conductive diamond electrode or a conductive diamond-like carbon electrode.

(3) In the analysis kit according to the above-described aspect, a composition of the magnetic metal nanoparticles may include at least one magnetic metal selected from a group consisting of iron, cobalt and nickel.

(4) In the analysis kit according to the above-described aspect, a composition of the magnetic metal nanoparticles may contain sulfur.

(5) In the analysis kit according to the above-described aspect, the reference electrode may be a silver-silver chloride electrode.

(6) In the analysis kit according to the above-described aspect, the counter electrode may be a carbon electrode, a noble metal electrode, a conductive diamond electrode, or a conductive diamond-like carbon electrode.

(7) An analysis method according to a second aspect is an analysis method which analyzes for a test substance contained in a test substance solution and includes: a first binding step of binding a test substance in a test substance solution to primary antibodies by contacting the test substance solution to a working electrode, a sensor having the working electrode, a reference electrode and a counter electrode, and the primary antibodies capable of binding to the test substance being fixed on a surface of the working electrode; a first washing step of washing the working electrode and removing the test substance solution attached to the working electrode; a second binding step of causing the working electrode and a dispersion liquid of magnetic metal nanoparticles in which the magnetic metal nanoparticles in which secondary antibodies that are bound to the test substance are fixed to surfaces thereof are dispersed to be in contact with each other and connecting the test substance with the magnetic metal nanoparticles by binding the test substance bound to the primary antibodies of the working electrode to the secondary antibodies; a second binding step of binding a test substance bound to the primary antibodies of the working electrode to secondary antibodies by contacting the working electrode to a dispersion liquid of magnetic metal nanoparticles having a secondary antibodies capable of binding to the test substance being fixed on a surface in order to connect the test substance and magnetic metal nanoparticles; a second washing step of washing the working electrode and removing the dispersion liquid of the magnetic metal nanoparticles; a magnetic field applying step of applying a magnetic field to the magnetic metal nanoparticles connected to the test substance in the presence of a solvent and bringing the magnetic metal nanoparticles into contact with the working electrode; a current amount measuring step of applying a voltage between the working electrode and the counter electrode to ionize the magnetic metal nanoparticles in the presence of a conductive solvent, and measuring an amount of current until a total amount of the magnetic metal nanoparticles is ionized; and a calculation step of calculating the amount of the magnetic metal nanoparticles from the amount of current and calculating an amount of the test substance from the amount of the magnetic metal nanoparticles.

(8) An analysis method according to a third aspect is an analysis method which analyzes for a test substance contained in a test substance solution and includes: a first binding step of binding a test substance in a test substance solution to primary antibodies by contacting the test substance solution to a working electrode, a sensor having the working electrode, a reference electrode and a counter electrode, and the primary antibodies capable of binding to the test substance being fixed on a surface of the working electrode; a washing step of washing the working electrode and removing the test substance solution attached to the working electrode; a second binding step of binding a test substance bound to the primary antibodies of the working electrode to secondary antibodies by contacting the working electrode to a dispersion liquid of magnetic metal nanoparticles having a secondary antibodies capable of binding to the test substance being fixed on a surface in order to connect the test substance and magnetic metal nanoparticles; an unconnected magnetic metal nanoparticle removing step of removing the magnetic metal nanoparticles which are not connected to the test substance from the surface of the working electrode or a vicinity thereof by an external magnetic field; a magnetic field applying step of applying a magnetic field to the magnetic metal nanoparticles connected to the test substance in the presence of a solvent and bringing the magnetic metal nanoparticles into contact with the working electrode; a current amount measuring step of applying a voltage between the working electrode and the counter electrode to ionize the magnetic metal nanoparticles in the presence of a conductive solvent, and measuring an amount of current until a total amount of the magnetic metal nanoparticles is ionized; and a calculation step of calculating the amount of the magnetic metal nanoparticles from the amount of current and calculating an amount of the test substance from the amount of the magnetic metal nanoparticles.

### Advantageous Effects of Invention

According to the present invention, it is possible to provide an analysis kit and an analysis method which are easy to operate and allow analysis for a test substance with high selectivity and high sensitivity.

### BRIEF DESCRIPTION OF DRAWINGS

FIG. 1 is a plan view of a sensor used in an analysis kit according to a first embodiment of the present invention.

FIG. 2 is a cross-sectional view taken along line II-II in FIG. 1.

FIG. 3 is a flowchart for explaining an analysis method according to a second embodiment of the present invention.

FIG. 4 is a conceptual diagram for explaining an analysis method according to the second embodiment of the present invention.

FIG. 5 is a flowchart for explaining an analysis method according to a third embodiment of the present invention.

FIG. 6 is a conceptual diagram for explaining an unconnected magnetic metal nanoparticle removing process of the analysis method according to the third embodiment of the present invention.

FIG. 7 is a graph plotting an antigen concentration and an amount of current required to ionize cobalt nanoparticles which are a label of an antigen in respective samples for antigen analysis used in Example 1.

FIG. 8 is a graph plotting an antigen concentration and an amount of current required to ionize cobalt nanoparticles which are a label of an antigen in respective samples for antigen analysis used in Example 2.

FIG. 9 is a graph plotting an antigen concentration and an amount of current required to ionize cobalt nanoparticles which are a label of an antigen in respective samples for antigen analysis used in Example 3.

FIG. 10 is a graph plotting an antigen concentration and an amount of current required to ionize cobalt nanoparticles which are a label of an antigen in respective samples for antigen analysis used in Example 4.

### DESCRIPTION OF EMBODIMENTS

The following, a constitution according to respective embodiments will be described with reference to the drawings. In the drawings used in the following description, to make features easy to understand, portions which become the features may be shown in an enlarged manner for the sake of convenience. Dimensional ratio or the like of respective constituents are not always the same as actual ones. In addition, materials, dimensions, and the like exemplified in the following description are examples, and the present invention is not limited thereto.

### First Embodiment

### [Analysis Kit]

The analysis kit of the embodiment is an analysis kit for analyzing a test substance contained in a test substance solution using an antigen-antibody reaction. The test substance is, for example, a biomaterial, and in particular, a protein or a metabolome.

The analysis kit of the embodiment includes a sensor and a dispersion liquid of the magnetic metal nanoparticles. The sensor has a function in which a test substance contained in a test substance solution is captured, and the magnetic metal nanoparticles function as a label for quantifying the captured test substance.

### (Sensor)

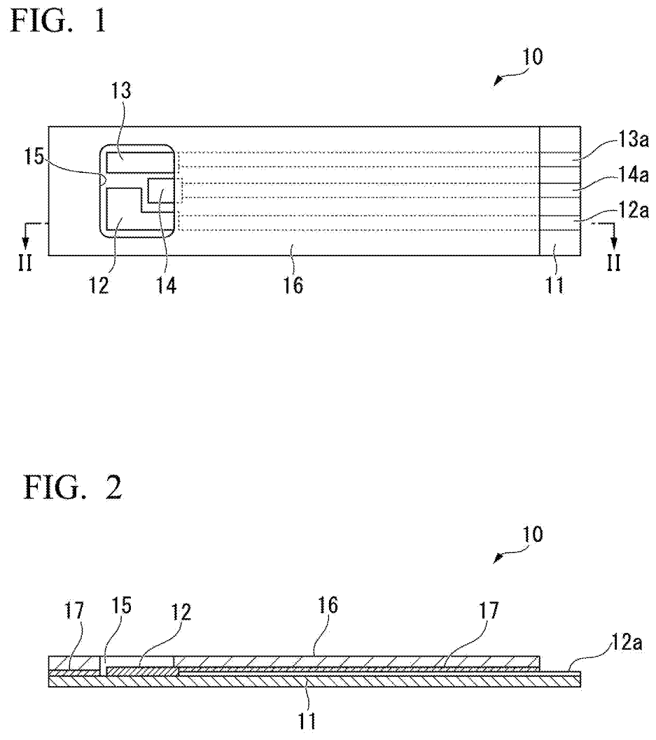

The sensor used in the analysis kit according to a first embodiment of the present invention will be described with reference to FIGS. 1 and 2. FIG. 1 is a plan view showing an embodiment of the sensor. FIG. 2 is a cross-sectional view taken along line II-II in FIG. 1.

The sensor 10 shown in FIG. 1 includes a first substrate 11; a working electrode 12, a counter electrode 13, and a reference electrode 14 provided in vicinity of one end portion on a surface (an upper surface in FIG. 2) of the first substrate 11; lead wires 12a, 13a and 14a formed on the first substrate 11 to be respectively connected to the working electrode 12, the counter electrode 13 and the reference electrode 14 and to extend to the other end portion of the first substrate 11; and a second substrate 16 adhered to the first substrate 11 and having a window 15 through which the working electrode 12, the counter electrode 13, and the reference electrode 14 are exposed. The second substrate 16 covers portions of the lead wires 12a, 13a, and 14a other than the vicinity of the other end portion of the first substrate 11.

The working electrode 12 is preferably a metal electrode, a carbon electrode, a conductive diamond electrode, or a conductive diamond-like carbon electrode (a DLC electrode). A copper electrode, a gold electrode, a platinum electrode, a palladium electrode, or the like can be used as the metal electrode. The metal electrode is preferably a noble metal electrode from a viewpoint of corrosion resistance. The carbon electrode is an electrode of carbon having conductivity such as graphite, and for example, a carbon printed electrode printed with a paste mainly composed of graphite can be used. A boron-doped diamond electrode doped with boron can be used as the conductive diamond electrode. The conductive diamond electrode is a crystalline carbon electrode having a diamond structure having sp³ bonds. The conductive DLC electrode is an amorphous carbon electrode mainly composed of carbon and hydrogen and in which sp³ bonds and sp² bonds are mixed. Either an n-type semiconductor DLC electrode doped with at least one element selected from a group consisting of nitrogen, phosphorus, arsenic, antimony and bismuth or a p-type semiconductor DLC electrode doped with an element selected from a group consisting of boron, gallium and indium can be used as the conductive DLC electrode.

The conductive diamond electrode and DLC electrode mainly composed of sp³-bound carbon undergo very few processes of adsorbing chemical substances which are oxidized or reduced by an electrochemical reaction. Thus, for example, it is difficult for an inner sphere oxidation-reduction reaction through adsorption to the electrode due to hydrogen, hydroxide, or ions caused by water to occur. As a result, since a noise current called a residual current becomes extremely lower, it is possible to detect the electrochemical reaction of the test substance to be detected with a high S/N ratio.

Primary antibodies are fixed to a surface (an upper surface in FIG. 2) of the working electrode 12. The primary antibodies are appropriately selected and used according to the test substance (an antigen) to be measured. Any primary antibodies can be used without particular limitation as long as they have a high affinity for the test substance to be measured and are capable of binding to the test substance (the antigen).

The counter electrode 13 is formed of a conductive material for an electrode which is usually used in a sensor for electrochemical measurement. As the counter electrode 13, for example, a carbon electrode, a noble metal electrode such as a platinum electrode, and a gold electrode, a conductive diamond electrode, or a conductive DLC electrode can be used.

As the reference electrode 14, for example, a silver-silver chloride electrode or a mercury-mercury chloride electrode can be used. The reference electrode 14 is preferably a silver-silver chloride electrode.

The first substrate 11 is a support body which supports the working electrode 12, the counter electrode 13, and the reference electrode 14. The first substrate 11 should have a physical strength sufficient for withstanding use in an electrochemical sensor.

When the working electrode 12 is an n-type semiconductor DLC electrode, the first substrate 11 is preferably an n-type crystalline silicon substrate. In addition, when the working electrode 12 is a p-type semiconductor DLC electrode, the first substrate 11 is preferably a p-type crystalline silicon substrate. Thus, it is difficult for an interface resistance such as a Schottky barrier to occur between the first substrate 11 and the working electrode 12.

As the second substrate 16, the same substrate as the first substrate 11 can be used. The first substrate 11 and the second substrate 16 are adhered by an adhesive 17.

### (Dispersion Liquid of Magnetic Metal Nanoparticles)

The dispersion liquid of the magnetic metal nanoparticles includes a solvent and magnetic metal nanoparticles dispersed in the solvent.

An aqueous solvent, an organic solvent, and a mixture thereof can be used as the solvent. Examples of the aqueous solvent include water and a buffer solution. Phosphate buffered saline (PBS) can be used as the buffer solution. Examples of the organic solvent include monohydric alcohols such as methanol, ethanol, 1-propanol and 2-propanol, and ketones such as acetone, methyl ethyl ketone and methyl isobutyl ketone.

The magnetic metal nanoparticles are preferably paramagnetic or superparamagnetic metal nanoparticles.

The magnetic metal nanoparticles preferably have an average particle diameter in an range of 1 nm to 50 nm, and more preferably in a range of 5 nm to 30 nm.

A composition of the magnetic metal nanoparticles preferably include at least one magnetic metal selected from a group consisting of iron, cobalt and nickel. One type of magnetic metal may be used individually or an alloy in which two or more types are combined may be used. The magnetic metal nanoparticles are preferably iron nanoparticles, cobalt nanoparticles, or nickel nanoparticles. One type of these magnetic metal nanoparticles may be used individually or a combination of two or more types may be used.

Secondary antibodies are fixed to surfaces of magnetic metal nanoparticles. The secondary antibodies are appropriately selected and used according to the test substance (the antigen) to be measured. Any secondary antibodies can be used without particular limitation as long as they have a high affinity for the test substance to be measured and are capable of binding to the test substance (the antigen).

A composition of the magnetic metal nanoparticles may contain sulfur. Sulfur may be attached to surfaces of the metal particle nanoparticles or may be inserted into a space between metal atoms. Sulfur has an effect of suppressing oxidation of metal nanoparticles. A content of sulfur in the magnetic metal nanoparticles is preferably in a range of 0.001% by mass to 0.5% by mass.

The dispersion solution of the magnetic metal nanoparticles may further contain a thickener, a surfactant, a dispersant, an antioxidant, and the like.

### Second Embodiment

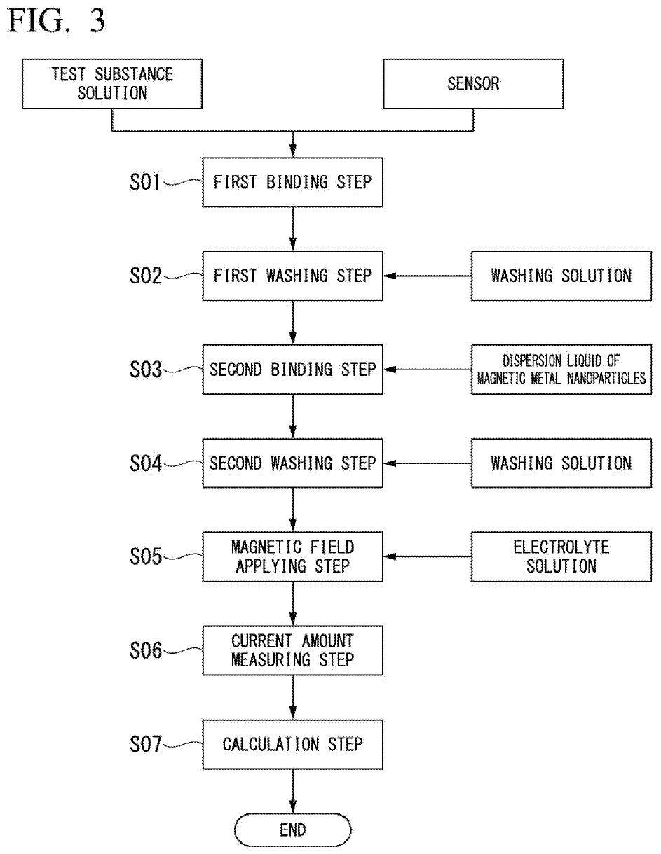

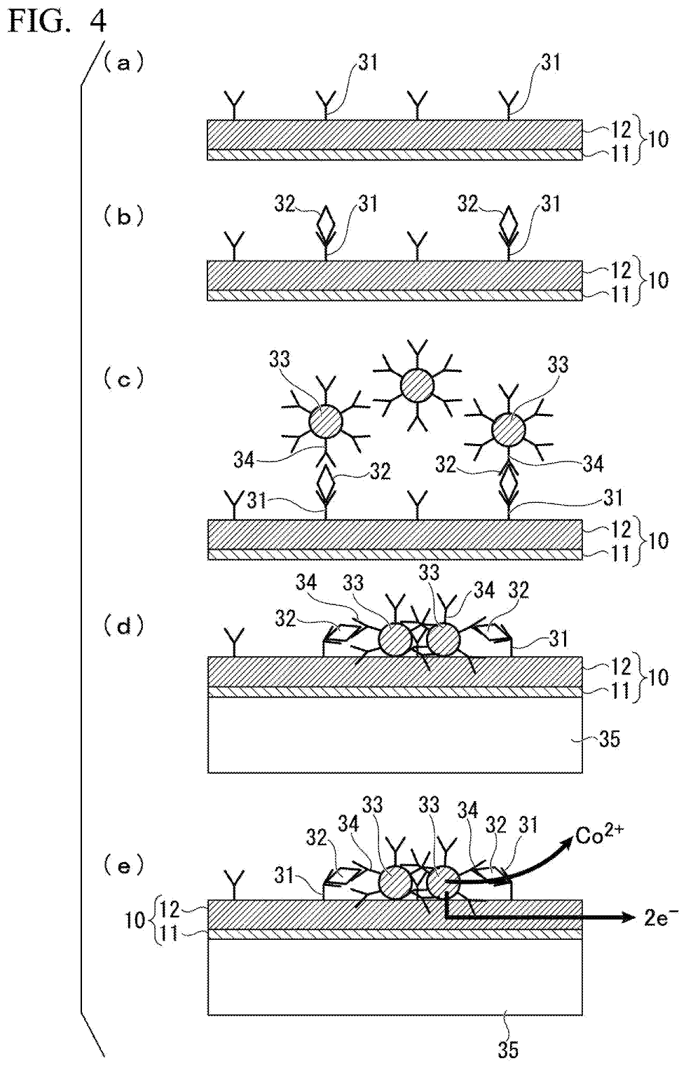

Next, an analysis method according to a second embodiment of the present invention will be described with reference to FIGS. 3 and 4.

FIG. 3 is a flowchart for explaining the analysis method according to the second embodiment of the present invention. FIG. 4 is a conceptual diagram for explaining an analysis method according to the second embodiment of the present invention.

As shown in FIG. 3, the analysis method of the embodiment includes a first binding step S01, a first washing step S02, a second binding step S03, a second washing step S04, a magnetic field applying step S05, a current amount measuring step S06, and a calculation step S07.

### (First Binding Step S01)

In the first binding step S01, the working electrode 12 of the sensor 10 as described above is brought into contact with the test substance solution. As shown in FIG. 4(a), primary antibodies 31 which can be selectively bound to the test substance that is a measurement target are fixed to the surface of the working electrode 12. Therefore, in the first binding step S01, as shown in FIG. 4(b), the primary antibodies 31 are captured with only the test substance 32 to be measured.

### (First Washing Step S02)

In the first washing step S02, the working electrode 12 is washed to remove the test substance solution attached to the working electrode 12. An aqueous solvent or an organic solvent can be used as a washing solution.

### (Second Binding Step S03)

In the second binding step S03, the dispersion liquid of the magnetic metal nanoparticles is brought into contact with the working electrode 12. As shown in FIG. 4(c), the secondary antibodies 34 which can be selectively bound to the test substance 32 which is a measurement target are fixed to surfaces of the magnetic metal nanoparticles 33. Therefore, as shown in FIG. 4(c), the test substance 32 and the secondary antibodies 34 are bound by the second binding step S03, and the magnetic metal nanoparticles 33 which become a label are connected to the test substance 32.

### (Second Washing Step S04)

In the second washing step S04, the working electrode 12 is washed to remove the dispersion liquid of the magnetic metal nanoparticles attached to the working electrode 12. The magnetic metal nanoparticles 33 which are not connected to the test substance 32 are removed by the second washing step S04. An aqueous solvent or an organic solvent can be used as a washing solution.

### (Magnetic Field Applying Step S05)

In the magnetic field applying step S05, a magnetic field is applied to the magnetic metal nanoparticles 33 connected to the test substance 32 in the presence of a solvent. The magnetic field is preferably applied in a direction in which the magnetic metal nanoparticles 33 are attracted from the back surface side of the working electrode 12. For example, as shown in FIG. 4(d), the magnetic field can be applied by disposing a magnet 35 on the back surface (the lower surface in FIG. 4(d)) side of the working electrode 12. A permanent magnet such as a neodymium magnet or an electromagnet which applies a magnetic field with a coil can be used as the magnet 35. Due to the magnetic field applying step S05, the magnetic metal nanoparticles 33 connected to the test substance 32 come into contact with the working electrode 12.

The solvent is not particularly limited as long as it can move the magnetic metal nanoparticles 33 to be brought into contact with the working electrode 12 by the applied magnetic field but is preferably a conductive solvent which can be used in the next current amount measuring step S06.

### (Current Amount Measuring Step S06)

In the current amount measuring step S06, a voltage is applied between the working electrode 12 and the counter electrode 13 to ionize (oxidize) the magnetic metal nanoparticles 33 in the presence of a conductive solvent, and an amount of current until the total amount of the magnetic metal nanoparticles 33 is ionized is measured. For example, when the magnetic metal nanoparticles 33 are cobalt nanoparticles, as shown in FIG. 4(d), the amount of current when the cobalt nanoparticles are dissolved as divalent ions is measured by applying a voltage between the working electrode 12 and the counter electrode 13. Specifically, the lead wires 12a, 13a, and 14a of the sensor 10 are connected to a potentiostat, and the amount of current flowing between the working electrode 12 and the counter electrode 13 is measured using a voltammetry method while a voltage is applied between the working electrode 12 and the counter electrode 13.

An electrolyte solution is preferably used as the conductive solvent. As an electrolyte of the electrolyte solution, a chloride such as potassium chloride, sodium chloride, and lithium chloride can be used. When the chloride ionizes the magnetic metal nanoparticles 33, the chloride has an effect of destroying an oxide film (a passive film) formed on surfaces of the magnetic metal nanoparticles 33, exposing the magnetic metal to the solution, and facilitating ionization. An aqueous solvent can be used as a solvent for the electrolyte solution. Examples of the aqueous solvent include water and a buffer solution. A chloride ion concentration of the electrolyte solution is preferably in a range of 0.05 mol/L or more and 1.0 mol/L or less.

### (Calculation Step S07)

In the calculation step S07, the amount of the magnetic metal nanoparticles 33 is obtained from the amount of current, and the amount of the test substance is calculated from the amount of the magnetic metal nanoparticles 33. The amount of current obtained in the current amount measuring step S06 correlates with the amount of the magnetic metal nanoparticles 33 (that is, the amount of the test substance). Therefore, the test substance contained in the test substance solution can be accurately quantified by creating a calibration curve using a sample containing a known amount of the test substance.

### Third Embodiment

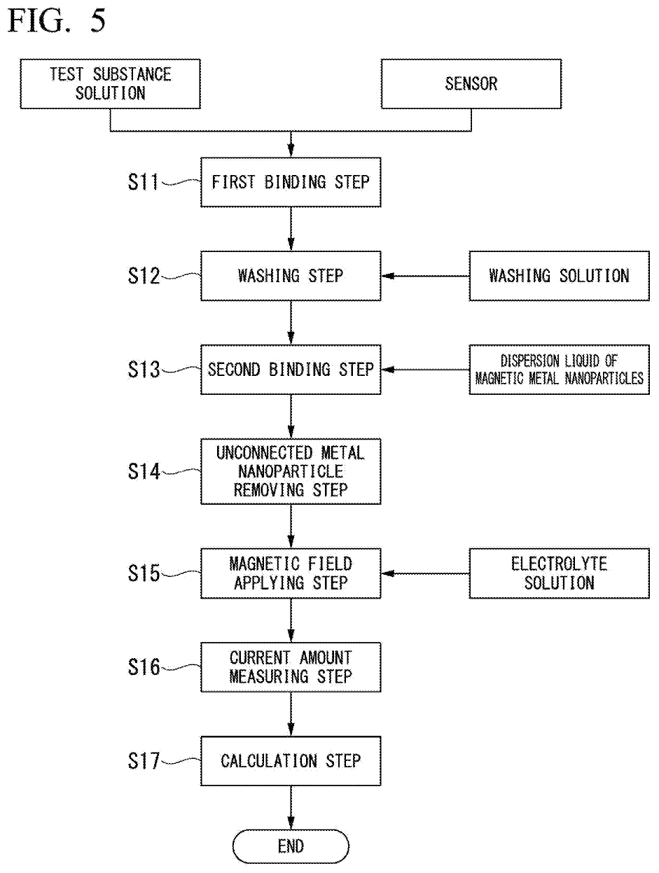

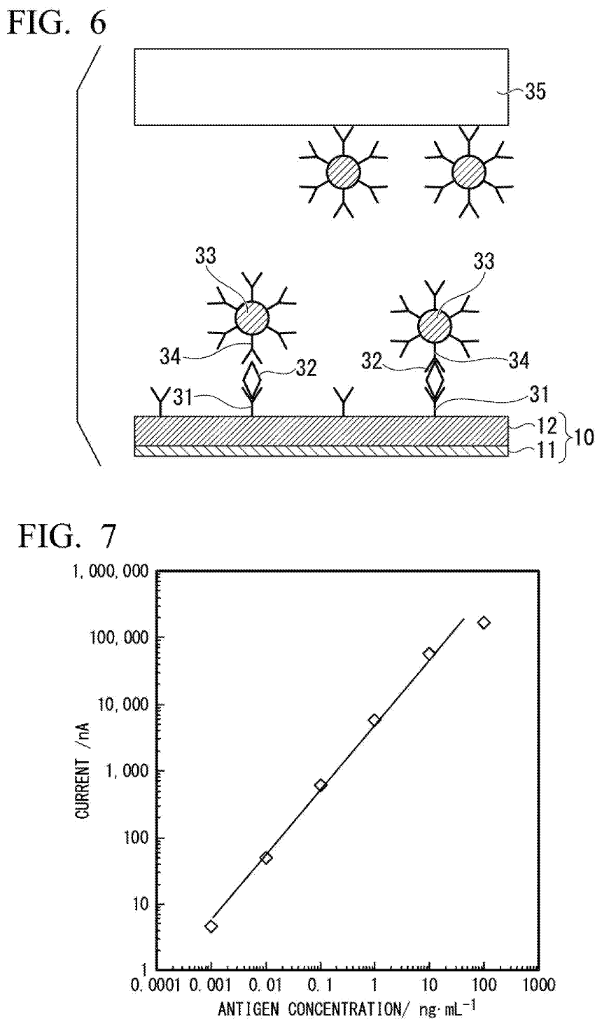

Next, an analysis method according to a third embodiment of the present invention will be described with reference to FIGS. 5 and 6.

FIG. 5 is a flowchart for explaining an analysis method according to the third embodiment of the present invention. FIG. 6 is a conceptual diagram for explaining an unconnected magnetic metal nanoparticle removing step in the analysis method according to the third embodiment of the present invention.

As shown in FIG. 5, the analysis method of the embodiment includes a first binding step S11, a washing step S12, a second binding step S13, an unconnected magnetic metal nanoparticle removing step S14, a magnetic field applying step S15, and a current amount measuring step S16, and a calculation step S17. The washing step S12 is the same as the above-described first washing step S02 of the first embodiment. The analysis method of the third embodiment is constituted in the same way as the analysis method of the first embodiment except that the unconnected magnetic metal nanoparticle removing step S14 is performed instead of the second washing step S04 of the first embodiment.

### (Unconnected Magnetic Metal Nanoparticle Removing Step S14)

In the unconnected magnetic metal nanoparticle removing step S14, the magnetic metal nanoparticles 33 which are not connected to the test substance 32 are removed from the surface of the working electrode 12 or the vicinity thereof by an external magnetic field. For example, as shown in FIG. 6, the magnet 35 is disposed on the surface of the working electrode 12 or in the vicinity thereof, and the magnetic metal nanoparticles 33 which are not connected to the test substance 32 are attached to the magnet 35 and removed by moving the magnet 35. A surface of the magnet 35 may be covered with a plastic film so that the magnet 35 and the magnetic metal nanoparticles 33 are not in direct contact with each other. The magnet 35 may be a permanent magnet or an electromagnet. A method for applying a magnetic field from the outside is not particularly limited, and a method using something other than a magnet may be used.

As described above, according to the analysis kit of the first embodiment, since the primary antibodies are fixed to the working electrode 12 of the sensor 10, the test substance contained in the test substance solution can be captured with high selectivity. Further, since the conductive diamond electrode or a conductive diamond-like carbon electrode (DLC electrode) is used as the working electrode 12 of the sensor 10, the electrochemical reaction of the magnetic metal nanoparticles can be detected with a high SN ratio.

Further, according to the analysis kit of the first embodiment, since the analysis kit has the dispersion liquid of the magnetic metal nanoparticles to which the secondary antibodies are fixed, the test substance captured with the primary antibodies can be analyzed with high sensitivity by using the magnetic metal nanoparticles as a label and quantifying the amount of the magnetic metal nanoparticles using an electrochemical method.

Further, according to the analysis method of the second embodiment and the third embodiment, since a magnetic field is applied to the magnetic metal nanoparticles connected to the test substance in the presence of a solvent and the magnetic metal nanoparticles are brought into contact with the working electrode, the test substance can be quantified using an electrochemical method without performing a conventional process for chemically dissolving a metal. Therefore, according to the analysis kit and analysis method of the present invention, it is easy to operate, and it is possible to analyze a test substance with high selectivity and high sensitivity.

### Examples

The following, the present invention will be described in more detail with reference to specific examples, but the present invention is not limited to these examples.

### Example 1

(1) Production of Sensor with Primary Antibodies

A sensor chip using a conductive DLC film as a working electrode, a carbon film created by screen printing of carbon paste as a counter electrode and a lead wire, and an Ag/AgCl film created by screen printing of pasted Ag/AgCl as a reference electrode was prepared. An unlabeled anti-goat IgG as the primary antibodies was fixed to the working electrode (having an electrode area S=0.0962 cm²) of the sensor chip, and a sensor with primary antibodies in which the primary antibodies are fixed to the surface of the working electrode was produced.

(2) Cobalt Nanoparticle Dispersion Liquid with Secondary Antibodies

4.60 mM of cobalt (II) sulfate tetrahydrate and 0.460 mM of trisodium citrate dehydrate were dissolved in 2 L of deionized water. 8.80 mM of sodium borohydride was added to the mixture and allowed to react for 10 minutes. The cobalt nanoparticles produced were separated using a neodymium magnet and washed several times with ethanol. After washing, the cobalt nanoparticles were dried at room temperature in a vacuum oven overnight. The dried cobalt nanoparticles were heat-treated at 450° C. for 1 hour under a mixed gas of hydrogen and nitrogen. An average particle diameter of the obtained cobalt nanoparticles was 18 nm.

The cobalt nanoparticles, ovalbumin (OA, grade III), anti-goat IgG, phosphate buffered saline (PBS), polyethylene glycol sorbitan monolaurate (Tween 20, nonionic surfactant) were mixed, and a cobalt nanoparticle dispersion liquid with the secondary antibodies in which anti-goat IgG (secondary antibodies) was fixed to surfaces of the cobalt nanoparticles was prepared. A concentration of the cobalt nanoparticles in the cobalt nanoparticle dispersion liquid with the secondary antibodies was 0.007 mass %. Polyclonal antibodies commercially available from Jackson Immunoresearch Laboratories were used as the anti-goat IgG.

### (3) Sample for Antigen Analysis

As samples for antigen analysis, as shown below, each of No. 1 to No. 6 with different antigen concentrations was prepared. The following No. 1 to No. 6 were prepared by mixing a PBS buffer solution containing 0.1% of Tween 20 with the goat IgG (the antigen) so that antigen concentrations became the following concentrations.

$$antigen concentration = 0.001 ng / mL \qquad No . 1$$

$$antigen concentration = 0.01 ng / mL \qquad No . 2$$

$$antigen concentration = 0.1 ng / mL \qquad No . 3$$

$$antigen concentration = 1 ng / mL \qquad No . 4$$

$$antigen concentration = 10 ng / mL \qquad No . 5$$

$$antigen concentration = 10 \; 0 ng / mL \qquad No . 6$$

### (4) Analysis of Antigen

For each of the samples for antigen analysis of No. 1 to No. 6 prepared in (3), 35 μL was accurately weighed, dropped onto the working electrode of the sensor with the primary antibodies prepared in the above-described (1), and then incubated for 40 minutes (the first binding step).

Next, the working electrode of the sensor with the primary antibodies was washed with a washing solution to wash away the sample for antigen analysis (the first washing step). PBS was used as the washing solution.

Next, 1 μL of the cobalt nanoparticle dispersion liquid with the secondary antibodies prepared in the above-described (2) was accurately weighed, dropped onto the working electrode of the sensor with the primary antibodies, and then incubated for 3 hours (the second binding step).

Next, the working electrode of the sensor with the primary antibodies was washed with a washing solution to wash away the cobalt nanoparticle dispersion liquid with the secondary antibodies (the second washing step). PBS was used as the washing solution.

Next, the lead wire of the sensor with the primary antibodies was connected to the potentiostat, and the sensor with the primary antibodies was accommodated in a plastic square container so that a back surface (a surface on the side opposite to the working electrode side) of the sensor with the primary antibodies was in contact with a bottom surface of the plastic square container. Then, an electrolyte solution in which 0.1 mol/L of potassium chloride was dissolved in PBS was injected into the plastic square container, and the sensor with the primary antibodies was immersed in the electrolyte solution. Then, a neodymium magnet was placed in close contact with the outside of a bottom portion of the plastic square container, and a magnetic field was applied to the working electrode of the sensor with the primary antibodies in a direction in which the cobalt nanoparticles are attracted from the back surface side of the sensor (the magnetic field applying step).

Next, a voltage was applied between the working electrode and the counter electrode using the potentiostat, and the amount of current flowing between the working electrode and the counter electrode was measured (the current amount measuring step).

FIG. 7 shows a graph plotting the antigen concentration and the amount of current flowing between the working electrode and the counter electrode (that is, the amount of current required to ionize the cobalt nanoparticles) in each of the samples for antigen analysis of No. 1 to No. 6. From the graph of FIG. 7, it was confirmed that there is a correlation between the current value and the antigen concentration of the sample for antigen analysis. Therefore, it was confirmed that the antigen (the test substance) contained in the test substance solution can be accurately quantified by creating a calibration curve (a current value-antigen concentration curve) using a sample containing a known amount of the antigen (the test substance).

### Example 2

(1) Production of Sensor with Primary Antibodies

A sensor chip using a gold vapor deposition film with a counter electrode and a lead wire patterned with a mask as a polyethylene terephthalate substrate, a conductive DLC film formed on a Si wafer as a working electrode and an Ag/AgCl film created by screen printing of pasted Ag/AgCl as a reference electrode was prepared. Unlabeled anti-8-hydroxydeoxyguanosine (anti-8-OHdG) antibodies were fixed as the primary antibodies on the working electrode (having an electrode area S-0.0962 cm²) of the sensor chip, and a sensor with primary antibodies in which the primary antibodies are fixed to the surface of the working electrode was produced.

(2) Cobalt Nanoparticle Dispersion Liquid with Secondary Antibodies

4.60 mM of cobalt (II) sulfate tetrahydrate and 0.460 mM of trisodium citrate dihydrate were dissolved in 2 L of deionized water. 8.80 mM of sodium borohydride was added to the mixture and allowed to react for 10 minutes. The cobalt nanoparticles produced were separated using a neodymium magnet and washed several times with ethanol. After washing, the cobalt nanoparticles were dried at room temperature in a vacuum oven overnight. The dried cobalt nanoparticles were heat-treated at 450° C. for 1 hour under a mixed gas of hydrogen and nitrogen. An average particle diameter of the obtained cobalt nanoparticles was 40 nm.

The cobalt nanoparticles, anti-8-hydroxydeoxyguanosine (anti-8-OHdG) antibodies, phosphate buffered saline (PBS), polyethylene glycol sorbitan monolaurate (Tween 20, nonionic surfactant) were mixed, and a cobalt nanoparticle dispersion liquid with the secondary antibodies in which anti-8-OHdG antibodies (the secondary antibodies) were fixed to surfaces of the cobalt nanoparticles was prepared. A concentration of the cobalt nanoparticles in the cobalt nanoparticle dispersion liquid with the secondary antibodies was 0.007 mass %. Monoclonal antibodies AA1005.1 commercially available from IMMUNDIAGNOSTIK GMBH were used as the anti-8-OHdG antibodies.

As samples for antigen analysis, as shown below, each of No. 1 to No. 6 with different antigen concentrations was prepared. The following No. 1 to No. 6 were prepared by mixing a PBS buffer containing 0.1% of Tween 20 with the 8-OHdG (the antigen) so that the antigen concentrations became the following concentrations.

$$antigen concentration = 0.01 ng / mL \qquad No . 1$$

$$antigen concentration = 0.1 ng / mL \qquad No . 2$$

$$antigen concentration = 1 ng / mL \qquad No . 3$$

$$antigen concentration = 10 ng / mL \qquad No . 4$$

$$antigen concentration = 10 \; 0 ng / mL \qquad No . 5$$

$$antigen concentration = 10 \; 00 ng / mL \qquad No . 6$$

### (4) Analysis of Antigen

For each of the samples for antigen analysis of No. 1 to No. 6 prepared in (3), 35 μL was accurately weighed, dropped onto the working electrode of the sensor with the primary antibodies prepared in the above-described (1), and then incubated for 40 minutes (the first binding step).

Next, the working electrode of the sensor with the primary antibodies was washed with a washing solution to wash away the sample for antigen analysis (the washing step). PBS was used as the washing solution.

Next, 1 μL of the cobalt nanoparticle dispersion liquid with the secondary antibodies prepared in the above-described (2) was accurately weighed, dropped onto the working electrode of the sensor with the primary antibodies, and then incubated for 3 hours (the second binding step).

Next, the lead wire of the sensor with the primary antibodies was connected to the potentiostat, and the sensor with the primary antibodies was accommodated in a plastic square container so that a back surface (a surface on the side opposite to the working electrode side) of the sensor with the primary antibodies was in contact with a bottom surface of the plastic square container. Then, an electrolyte solution in which 0.1 mol/L of potassium chloride was dissolved in PBS was injected into the plastic square container, and the sensor with the primary antibodies was immersed in the electrolyte solution. Then, a neodymium magnet was placed on the surface of the working electrode of the sensor substrate with the primary antibodies, and the unconnected cobalt nanoparticles with the secondary antibodies were attached to the neodymium magnet and then removed (the unconnected magnetic metal nanoparticle removing step).

Next, the neodymium magnet was placed in close contact with the outside of a bottom portion of the plastic square container, and a magnetic field was applied to the working electrode of the sensor with the primary antibodies in a direction in which the cobalt nanoparticles are attracted from the back surface side of the sensor (the magnetic field applying step).

Next, a voltage was applied between the working electrode and the counter electrode using the potentiostat, and the amount of current flowing between the working electrode and the counter electrode was measured (the current amount measuring step).

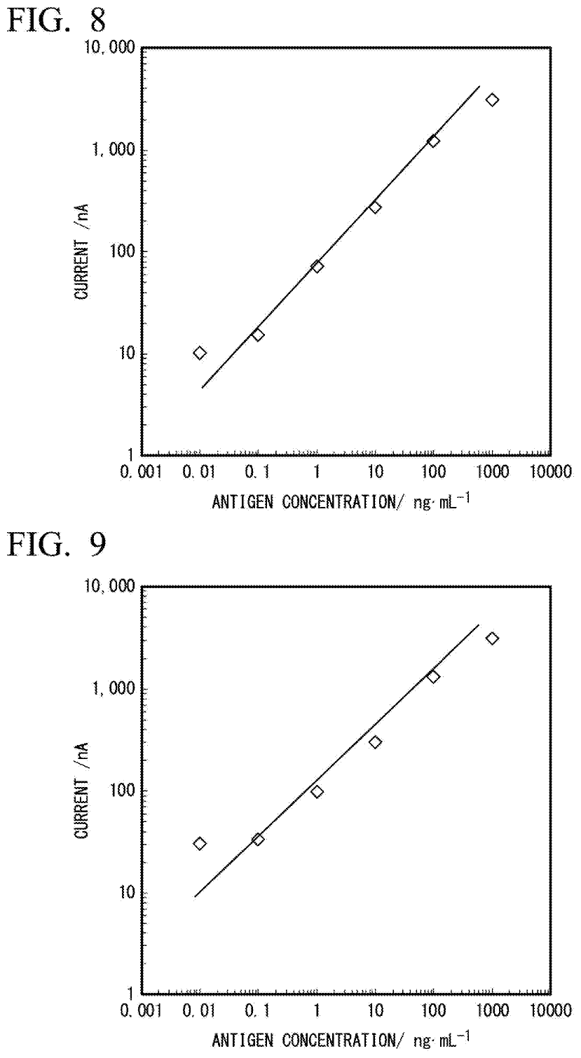

FIG. 8 shows a graph plotting the antigen concentration and the amount of current flowing between the working electrode and the counter electrode (that is, the amount of current required to ionize the cobalt nanoparticles) in each of the samples for antigen analysis of No. 1 to No. 6. From the graph of FIG. 8, it was confirmed that there is a correlation between the current value and the antigen concentration of the sample for antigen analysis. Therefore, it was confirmed that the antigen (the test substance) contained in the test substance solution can be accurately quantified by creating a calibration curve (a current value-antigen concentration curve) using a sample containing a known amount of the antigen (the test substance).

### Example 3

A graph plotting the antigen concentration of each of the samples for antigen analysis and the amount of current required to ionize the cobalt nanoparticles as the label of the antigen was created in the same manner as in Example 2, except that the working electrode of the sensor with the primary antibodies was a carbon printed electrode. The result is shown in FIG. 9. From the graph of FIG. 9, it was confirmed that there is a correlation between the current value and the antigen concentration of the sample for antigen analysis. Thus, it was confirmed that even when the carbon printed electrode was used as the working electrode, it was possible to accurately quantify the antigen (test substance) contained in the test substance solution.

### Example 4

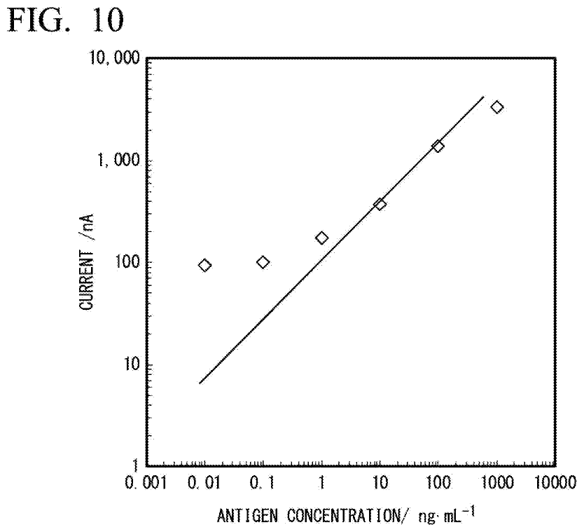

A graph plotting the antigen concentration of each of the samples for antigen analysis and the amount of current required to ionize the cobalt nanoparticles as the label of the antigen was created in the same manner as in Example 2, except that the working electrode of the sensor with the primary antibodies was a gold vapor deposition electrode formed by vapor deposition. The result is shown in FIG. 10. From the graph of FIG. 10, it was confirmed that there is a correlation between the current value and the antigen concentration of the sample for antigen analysis. Thus, it was confirmed that even when the gold vapor deposition electrode was used as the working electrode, it was possible to accurately quantify the antigen (test substance) contained in the test substance solution.

### REFERENCE SIGNS LIST

10 Sensor
11 First substrate
12 Working electrode
13 Counter electrode
14 Reference electrode
12a, 13a, 14a Lead wire
15 Window
16 Second substrate
17 Adhesive
31 Primary antibody
32 Test substance
33 Magnetic metal nanoparticle
34 Secondary antibody
35 Magnet

1. An analysis method which analyzes for a test substance contained in a test substance solution, the method comprising:
a first binding step of binding a test substance in a test substance solution to primary antibodies, the primary antibodies capable of binding to the test substance being fixed on a surface of the working electrode by contacting the test substance solution to a working electrode of the sensor of an analysis kit, the analysis kit comprising:
a sensor having a working electrode, a reference electrode and a counter electrode, primary antibodies being fixed to a surface of the working electrode of the sensor, and
a dispersion liquid of magnetic metal nanoparticles including solvent and magnetic metal nanoparticles dispersed in the solvent, secondary antibodies being fixed to surfaces of the magnetic metal nanoparticles;
a first washing step of washing the working electrode and removing the test substance solution attached to the working electrode;
a second binding step of binding a test substance bound to the primary antibodies of the working electrode to secondary antibodies by contacting the working electrode to a dispersion liquid of magnetic metal nanoparticles having a secondary antibodies capable of binding to the test substance being fixed on a surface in order to connect the test substance and magnetic metal nanoparticles;
a second washing step of washing the working electrode and removing the dispersion liquid of the magnetic metal nanoparticles;
a magnetic field applying step of applying a magnetic field to the magnetic metal nanoparticles connected to the test substance in the presence of a solvent and bringing the magnetic metal nanoparticles into contact with the working electrode;
a current amount measuring step of applying a voltage between the working electrode and the counter electrode to ionize the magnetic metal nanoparticles in the presence of a conductive solvent, and measuring an amount of current until a total amount of the magnetic metal nanoparticles is ionized; and
a calculation step of calculating the amount of the magnetic metal nanoparticles from the amount of current and calculating an amount of the test substance from the amount of the magnetic metal nanoparticles.

2. An analysis method which analyzes for a test substance contained in a test substance solution, the method comprising:
a first binding step of binding a test substance in a test substance solution to primary antibodies, and the primary antibodies capable of binding to the test substance being fixed on a surface of the working electrode by contacting the test substance solution to a working electrode of the sensor of an analysis kit, the analysis kit comprising:
a sensor having a working electrode, a reference electrode and a counter electrode, primary antibodies being fixed to a surface of the working electrode of the sensor, and
a dispersion liquid of magnetic metal nanoparticles including solvent and magnetic metal nanoparticles dispersed in the solvent, secondary antibodies being fixed to surfaces of the magnetic metal nanoparticles;
a washing step of washing the working electrode and removing the test substance solution attached to the working electrode;
a second binding step of binding a test substance bound to the primary antibodies of the working electrode to secondary antibodies by contacting the working electrode to a dispersion liquid of magnetic metal nanoparticles having a secondary antibodies capable of binding to the test substance being fixed on a surface in order to connect the test substance and magnetic metal nanoparticles;
an unconnected magnetic metal nanoparticle removing step of removing the magnetic metal nanoparticles which are not connected to the test substance from the surface of the working electrode or a vicinity thereof by an external magnetic field;
a magnetic field applying step of applying a magnetic field to the magnetic metal nanoparticles connected to the test substance in the presence of a solvent and bringing the magnetic metal nanoparticles into contact with the working electrode;
a current amount measuring step of applying a voltage between the working electrode and the counter electrode to ionize the magnetic metal nanoparticles in the presence of a conductive solvent, and measuring an amount of current until a total amount of the magnetic metal nanoparticles is ionized; and
a calculation step of calculating the amount of the magnetic metal nanoparticles from the amount of current and calculating an amount of the test substance from the amount of the magnetic metal nanoparticles.

3. The analysis method according to claim 1, wherein the working electrode is a carbon electrode, a metal electrode, a conductive diamond electrode or a conductive diamond-like carbon electrode.

4. The analysis method according to claim 2, wherein the working electrode is a carbon electrode, a metal electrode, a conductive diamond electrode or a conductive diamond-like carbon electrode.

5. The analysis method according to claim 1, wherein the magnetic metal nanoparticles include at least one magnetic metal selected from a group consisting of iron, cobalt and nickel.

6. The analysis method according to claim 2, wherein the magnetic metal nanoparticles include at least one magnetic metal selected from a group consisting of iron, cobalt and nickel.

7. The analysis method according to claim 1, wherein the magnetic metal nanoparticles contain sulfur.

8. The analysis method according to claim 2, wherein the magnetic metal nanoparticles contain sulfur.

9. The analysis method according to claim 1, wherein the reference electrode is a silver-silver chloride electrode.

10. The analysis method according to claim 2, wherein the reference electrode is a silver-silver chloride electrode.

11. The analysis method according to claim 1, wherein the counter electrode is a carbon electrode, a noble metal electrode, a conductive diamond electrode or a conductive diamond-like carbon electrode.

12. The analysis method according to claim 2, wherein the counter electrode is a carbon electrode, a noble metal electrode, a conductive diamond electrode or a conductive diamond-like carbon electrode.

13. The analysis method according to claim 1, wherein an electrolyte of the conductive solvent is chloride.

14. The analysis method according to claim 2, wherein an electrolyte of the conductive solvent is chloride.
